(19) United States
(12) Patent Application Publication
(10) Pub. No.: US 2012/0059372 A1
(43) Pub. Date: Mar. 8, 2012

(54) Electrosurgical Instrument

(76) Inventor: Kristin D. Johnson, Louisville, CO (US)

(21) Appl. No.: 12/876,731

(22) Filed: Sep. 7, 2010

(51) Int. Cl. A61B 18/14

(52) U.S. Cl. 606/45

(57) ABSTRACT

An end effector assembly for an electrosurgical instrument is provided. The end effector assembly has a pair of first and second jaw members including respective seal having a width. Each of the seal plates adapted to connect to an energy source. The first and second jaw members are operable in a first mode of operation for treating tissue and a second mode of operation for separating tissue. The width of the seal plate of the first jaw member is smaller than the width of the seal plate of the second jaw member. In the second mode of operation the seal plate of the first jaw member is independently activatable from the seal plate of the second jaw member to facilitate the separation of tissue when the first and second jaw members are in one of the open and clamping positions.

### BACKGROUND

1. Technical Field

The present disclosure relates to an electrosurgical instrument and, more particularly, to an electrosurgical instrument configured to dissect, seal or otherwise treat tissue.

2. Background of Related Art

Electrosurgical instruments, e.g., electrosurgical forceps (open type or closed type, i.e., suitable for a laparoscopic procedure), are well known in the medical arts and typically include an end effector assembly including jaw members configured to manipulate tissue (e.g., grasp and seal tissue). Typically, the electrosurgical forceps utilizes both mechanical clamping action and electrical energy to effect hemostasis by heating the tissue and blood vessels to coagulate, cauterize, seal, desiccate, and/or fulgurate tissue.

In certain instances, it may prove advantageous to cut or dissect tissue that has been electrosurgically treated, e.g., sealed. In such instances, a cutting element, e.g., a knife blade, may be configured to translate through a knife channel that is disposed on one or both of the jaw members. As can be appreciated, incorporating the knife blade into the electrosurgical instrument may increase manufacturing costs of the electrosurgical instrument. In addition, manufacturing tolerances typically associated with the placement of the knife channel on one or both of the jaw members need to be kept at a minimum. That is, the knife blade needs to be substantially aligned with the knife channel during the manufacture of the end effector and/or jaw members. As can be appreciated, if the knife blade and knife channel are not substantially aligned with each other, then during translation of the knife blade through the knife channel, the knife blade may contact the knife channel, which, in turn, may lead to tissue not being effectively severed.

In addition to electrosurgical instruments, ultrasonic instruments may be utilized to treat tissue. Conventional ultrasonic instruments, e.g., an ultrasonic dissector, typically include an end effector assembly including jaw members configured to manipulate tissue (e.g., grasp and seal tissue). Typically, the ultrasonic dissector utilizes both mechanical clamping action and ultrasonic energy to effect hemostasis by heating the tissue and blood vessels to coagulate, cauterize, seal, cut, dissect, desiccate, and/or fulgurate tissue. While ultrasonic instruments may effectively treat and, subsequently, dissect tissue, ultrasonic instruments are typically not configured to articulate and/or “flex.” As can be appreciated, this limits their use in the surgical environment.

### SUMMARY

The present disclosure provides an end effector assembly for an electrosurgical instrument. The end effector assembly has a pair of first and second jaw members including respective seal plates having a width. Each of the seal plates is adapted to connect to an energy source. One or both of the first and second jaw members may be movable relative to the other jaw member from an open position, wherein the first and the second jaw members are disposed in spaced relation relative to one another, to a clamping position, wherein the first and second jaw members cooperate to grasp tissue therebetween. The first and second jaw members are operable in two modes of operation, a first mode of operation for treating tissue and a second mode of operation for separating tissue. The seal plate of the first jaw member includes a width that is smaller than a width of the seal plate of the second jaw member. In the second mode of operation, the seal plate of the first jaw member is independently activatable from the seal plate of the second jaw member to facilitate the separation of tissue when the first and second jaw members are in one of the open and clamping positions.

The present disclosure provides a system for performing an electrosurgical procedure. The system includes an energy source that is configured to function in two or more modes of operation, a first mode of operation for treating tissue and a second mode of operation for dissecting tissue. The system includes an electrosurgical forceps that includes a handle having a shaft that extends therefrom and defines a longitudinal axis therethrough. An end effector assembly operatively connected to a distal end of the shaft and has a pair of first and second jaw members including respective seal plates having a width. Each of the seal plates is adapted to connect to an energy source. One or both of the first and second jaw members are movable relative to the other jaw member from an open position, wherein the first and the second jaw members are disposed in spaced relation relative to one another, to a clamping position, wherein the first and second jaw members cooperate to grasp tissue therebetween. The first and second jaw members are operable the first mode of operation for treating tissue and the second mode of operation for separating tissue. The seal plate of the first jaw member includes a width that is smaller than a width of the seal plate of the second jaw member. In the second mode of operation, the seal plate of the first jaw member is independently activatable from the seal plate of the second jaw member to facilitate the separation of tissue when the first and second jaw members are in one of the open and clamping positions.

The present disclosure also provides a method for performing an electrosurgical procedure. The method includes positioning tissue between first and second jaw members of an electrosurgical instrument. The first and second jaw members are operable in two modes of operation, a first mode of operation for treating tissue and a second mode of operation for separating tissue. The seal plate of the first jaw member includes a width that is smaller than a width of the seal plate of the second jaw member. The method includes closing the first and second jaw members such that the tissue is clamped therebetween. Transmitting electrosurgical energy in the first mode of operation to the first and second jaw members for electrosurgically treating tissue is a step of the method. And, transmitting electrosurgical energy in the second mode of operation to the seal plate of the first jaw member for dissecting the electrosurgically treated tissue is another step of the method.

In an embodiment, in the second mode of operation, the seal plate of the first jaw member is independently activatable from the seal plate of the second jaw member to facilitate the separation of tissue when the first and second jaw members are in one of the open and clamping positions.

### BRIEF DESCRIPTION OF THE DRAWINGS

Embodiments of the presently disclosed specimen retrieval apparatus are described hereinbelow with reference to the drawings wherein:

FIG. 1 is a left, perspective view of an electrosurgical instrument including an end effector having jaw members according to an embodiment of the present disclosure;

FIG. 2 is an enlarged, left perspective view of the indicated area of detail of FIG. 1 with a dissecting jaw member adjacent tissue;

FIGS. 3A-3C are front views of the jaw members depicted in FIG. 2 illustrating various alignment configurations thereof;

FIG. 4 is side view of jaw members depicted in FIG. 2 with tissue positioned across the dissecting jaw member; and

FIG. 5 is side view of jaw members depicted in FIG. 2 with tissue positioned across the dissecting jaw member with the jaw members in a clamping position.

### DETAILED DESCRIPTION OF THE EMBODIMENTS

Detailed embodiments of the present disclosure are disclosed herein; however, the disclosed embodiments are merely examples of the disclosure, which may be embodied in various forms. Therefore, specific structural and functional details disclosed herein are not to be interpreted as limiting, but merely as a basis for the claims and as a representative basis for teaching one skilled in the art to variously employ the present disclosure in virtually any appropriately detailed structure.

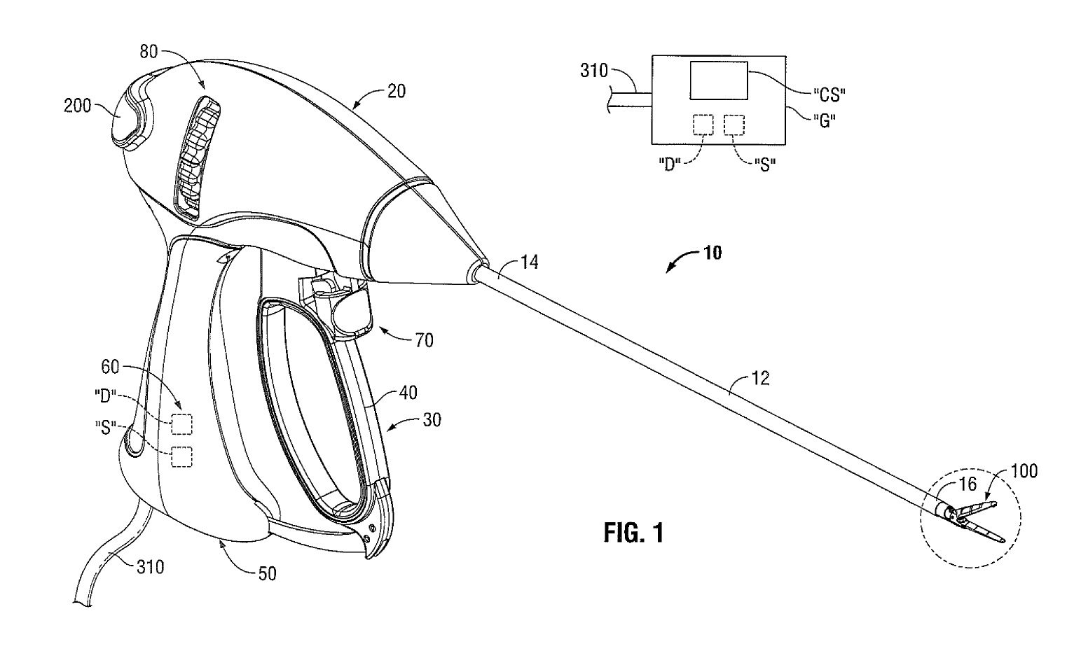

Turning now to FIGS. 1-4, and initially with reference to FIG. 1, an electrosurgical instrument, e.g., an electrosurgical forceps 10 (forceps 10), that includes an end effector 100 according to an embodiment of the present disclosure is shown. Forceps 10 is operatively and selectively coupled to an electrosurgical generator (generator “G”) for performing an electrosurgical procedure (FIG. 1). In some instances, the forceps 10 may be battery-powered. For purposes herein, an electrosurgical procedure may include sealing, cutting, dissecting, cauterizing, coagulating, desiccating, and/or fulgurating tissue all of which may employ RF energy. The generator “G” is configured for monopolar and bipolar modes of operation. The generator “G” may include or is in operative communication with a control system “CS” (FIG. 1) that may include one or more processors in operative communication with one or more control modules that are executable on the processor. The control module (not explicitly shown) may be configured to instruct one or more modules to transmit electrosurgical energy, which may be in the form of a wave or signal/pulse, via one or more cables (e.g., a cable 310) to one or both jaw members 110 and 120 of an end effector 100.

Briefly, forceps 10 is configured for use with various surgical procedures and includes a housing 20. A shaft 12 extends distally from the housing 20 and defines a longitudinal axis “A-A” therethrough. In the drawings and in the descriptions that follow, the term “proximal,” as is traditional, will refer to the end of the forceps 10 that is closer to the user, while the term “distal” will refer to the end that is farther from the user. The shaft has a distal end 16 configured to mechanically engage the end effector assembly 100 and a proximal end 14 that mechanically engages the housing 20. In certain instances, the shaft 12 may be configured to bend or articulate. For example, shaft 12 may be resilient or portion thereof may include an articulating member.

A handle assembly 30 includes a fixed handle 50 and a movable handle 40. Fixed handle 50 is integrally associated with housing 20 and handle 40 is movable relative to fixed handle 50. In certain embodiments, movable handle 40 of handle assembly 30 may be operably coupled to a drive assembly (not shown), which together may be configured to cooperate to impart movement of one or both of jaw members 110 and 120 to move from an open position, wherein the jaw members 110 and 120 are disposed in spaced relation relative to one another, to a clamping or closed position, wherein the jaw members 110 and 120 cooperate to grasp tissue therebetween. Although the figure drawings depict a forceps 10 for use in connection with endoscopic surgical procedures, the present disclosure may be used for more traditional open surgical procedures. The open version of the forceps may also include the same or similar operating components and features as described below.

For a more detailed description of the housing 20, shaft 20, handle assembly 30 (including movable and fixed handles 40 and 50, respectively), rotating assembly 80, trigger assembly 70 and electrosurgical cable 310 (including line-feed configurations and/or connections), reference is made to commonly-owned U.S. Pat. No. 7,150,097 to Sremcich filed Jun. 13, 2003.

With continued reference to FIG. 1, one or more buttons or switches 60 are operably disposed on the forceps 10. More particularly, and in the illustrated embodiment, two switches “D” and “S” are shown operably disposed on the fixed handle 50. In certain embodiments, it may prove advantageous to provide the switches 60 on the generator “G”. This of course will depend on the contemplated uses of a manufacturer. Switches “D” and “S” are in operative communication with the generator “G” and/or control system “CS” and are configured to place the forceps 10 in one or more modes of operation. More particularly, switch “S” is configured to place the forceps 10 in a first mode of operation for treating tissue, e.g., sealing tissue, and switch “D” is configured to place the forceps 10 in a second mode of operation for separating tissue, e.g., dissecting tissue.

In the first mode of operation the generator “G” including control system “CS” and the forceps 10 are configured to fuse, seal, coagulate and/or fulgurate tissue. To this end, the jaw members 110 and 120 are configured to function in a bipolar mode of operation. That is, respective seal plates 118 and 128 of jaw members 110 and 120 are both active, include opposing polarities and are configured to transmit electrosurgical energy, e.g., current, therebetween. In the second mode of operation the generator “G” including control system “CS” and the forceps 10 are configured to dissect, cut, severe and/or transect tissue. To this end, the jaw members 110 and 120 are configured to function in a monopolar mode of operation. That is, seal plate 128 is active, seal plate 118 is inactive or neutral (and/or is highly resistive to current flow), and seal plate 128 is configured to transmit electrosurgical energy, e.g., current, to tissue. In the monopolar mode of operation, a return pad or electrode is positioned on a patient and is configured to provide a return path for the current back to the generator “G”.

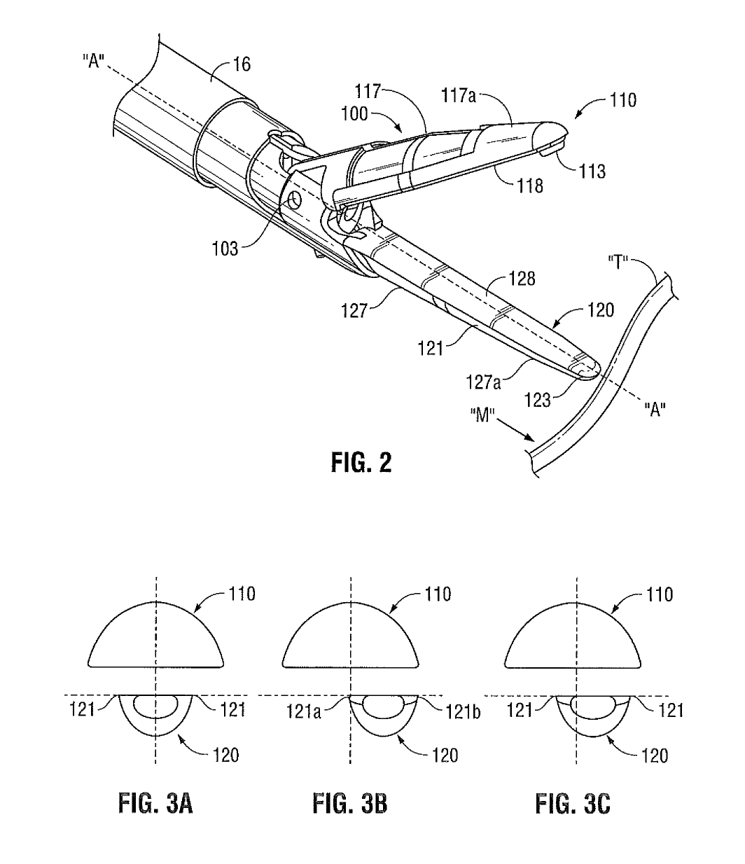

With reference to FIG. 2, an embodiment of end effector assembly 100 including jaw members 110 and 120 is illustrated. In the illustrated embodiment, jaw members 110 and 120 are of the unilateral type. That is, jaw member 110 is movable, e.g., pivotable, with respect to jaw member 120. Alternatively, jaw members 110 and 120 may be of the bilateral type. That is, each of the jaw members 110 and 120 are movable with respect to each other, In an embodiment, to facilitate pivoting the jaw member 110 with respect to jaw member 120, a pivot pin 103 couples the jaw members 110 and 120 to the distal end 16 of the shaft 12, as best seen in FIG. 2. Jaw members 110 and 120, and operative components associated therewith, may be formed from any suitable material, including but not limited to metal, metal alloys, plastic, plastic composites, and so forth.

Continuing with reference to FIG. 2, jaw member 110 is shown including a jaw housing 117 having a width of suitable proportion, i.e., a width that is suitable to support the seal plate 118. Electrically conductive seal plate 118 is operably supported on and secured to jaw housing 117. More particularly, a distal end 117a of jaw member 110 may be configured to securely engage the electrically conductive seal plate 118 or, with respect to a monolithic jaw member, form the seal plate 118.

One or more insulative or non-conductive standoffs 113 (one insulative standoff is shown) made of any suitable material, e.g., plastic, ceramic, etc., is operably disposed on the seal plate 118. More particularly, the insulative standoff 113 is operably disposed on a seal surface of the seal plate 118 at a distal end thereof; as best seen in FIG. 2. Insulative standoff 113 may be secured to the seal surface of the seal plate 118 by one or more suitable securement methods, e.g., an adhesive. In the illustrated embodiment, a “pocket” is etched in the seal surface during the manufacture process thereof, a bead of adhesive is placed in the “pocket” and the insulative standoff 113 is positioned therein. Other securement methods are contemplated.

The insulative standoff 113 is configured to contact a distal tip of the seal plate 128 when the jaw members 110 and 120 are in the clamping position such that a gap distance of suitable proportion is present between the seal surface of the seal plate 118 and a seal surface of a seal plate 128 of the jaw member 120. As a result thereof, the jaw members 118 and 128 only contact at their respective tips. Having the insulative standoff 113 positioned at the distal end of the seal plate minimizes any negative effects that may be associated with a non-conductive member being positioned on the seal surface of the seal plate 128. That is, having a portion of the seal surface of the seal plate 118 that does not conduct electrosurgical energy may compromise a tissue seal, e.g., the tissue seal may not be uniform and/or consistent across a length thereof. A uniform and/or consistent tissue seal is important, especially in the instance where one jaw member, e.g., jaw member 120, includes a seal plate 128 having a width that is smaller or “finer” than the other seal plate, e.g., seal plate 118. That is, the width of the tissue seal achieved with the jaw members 110 and 120 of the present disclosure is smaller (and thus the overall area of the tissue seal is smaller) than widths of tissue seals typically achieved by conventional jaw members.

Unlike conventional electrosurgical forceps that include end effectors having jaw members with seal plates having the same width, seal plates 118 and 128 of respective jaw members 110 and 120 of end effector 100 have different widths. More particularly, to facilitate separating tissue during the second mode of operation, the seal plate 128 of the jaw member 120 includes a width that is small in comparison to the width of the seal plate 118 of the jaw member 110. That is, seal plate 128 of the jaw member 120 is smaller or “finer” than the jaw member seal plate 118 of the 110 (see FIG. 2 in combination with FIGS. 3A-3C). For illustrative purposes, a width of the jaw housing 127 of the jaw member 120 is also illustrated as being smaller than a width of the jaw housing 117 of the jaw member 110. In some embodiments, it may prove advantageous to have the jaw housing 117 and 127 with the same widths and the seal plates 118 and 128 with different widths. The specific configuration, e.g., widths, of the jaw housing 117 and 127 may be varied for a specific surgical procedure, specific manufacturer requirement, etc. In accordance with an embodiment of the present disclosure, seal plate 118 of the jaw member 110 (the larger jaw) includes a width that is approximately 1 mm to 2 mm larger than the width of the seal plate 128 of the jaw member 120 (the smaller or “finer” jaw member). Keeping the width of the seal plate 128 1 mm to 2 mm smaller than the width of the seal plate 117 improves visualization and dissection capabilities for the end user, e.g., a surgeon. In the illustrated embodiment, seal plate 128 of the jaw member 120 includes a width that ranges from about 1 mm to about 3.4 mm and seal plate 118 of the jaw member 120 includes a width that ranges from about 3.5 mm to about 5 mm.

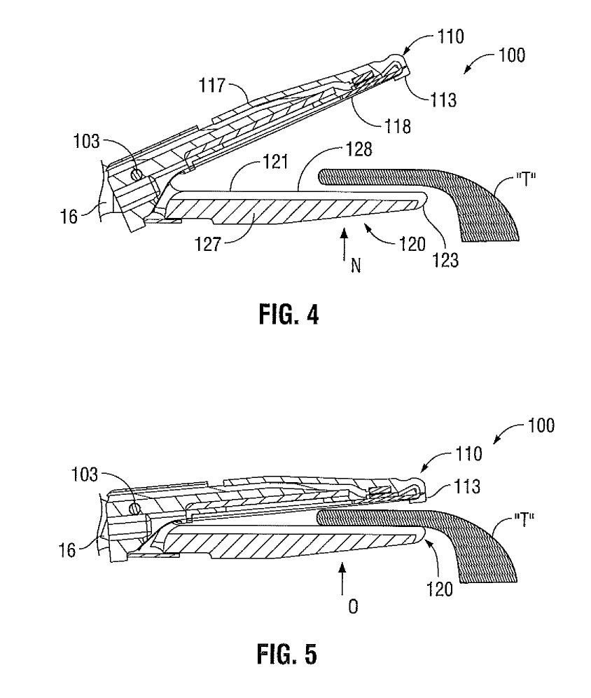

Similar to jaw member 110, jaw member 120 includes a jaw housing 127 having a distal end 127a that is configured to support seal plate 128 (FIG. 2). Unlike jaw member 110, jaw member 120 includes a seal plate 128 that includes a peripheral edge 121 that extends along a side surface of the jaw housing 127 to a distal tip 123 thereof, see FIGS. 2, 4 and 5. The peripheral edge 121 including the distal tip 123 is configured to separate tissue, e.g., dissect tissue, when the jaw members 110 and 120 are in either the open position (FIGS. 2 and 4) or the closed position (FIG. 5) and when tissue is positioned adjacent thereto. More particularly, and in one particular embodiment, when switch “D” is activated, the forceps 10 is configured to operate in the second mode of operation, e.g., a monopolar mode of operation. In the second mode of operation, the generator “G” transmits electrosurgical energy to the seal plate 128 including the peripheral edge 121 and the distal tip 123 such that a user may dissect tissue that has been electrosurgically treated.

Referring to FIGS. 3A-3C, to facilitate treating and/or separating tissue, jaw members 110 and 120 may be aligned along their center lines, i.e., centrally aligned along a common axis, e.g., longitudinal axis “A-A” (FIGS. 2 and 3A); aligned along a right or left portion of the peripheral edge 121 (FIG. 3B); or aligned somewhere therebetween (FIG. 3C). In the illustrated embodiment, the jaw members 110 and 120 are centrally aligned along the longitudinal axis “A-A,” as best seen in FIGS. 2 and 3A. Alignment along the longitudinal axis “A-A” facilitates dissecting tissue from either side of the forceps 10. In the embodiment where jaw members 110 and 120 are aligned along the right or left portion the peripheral edge 121 (see FIG. 3B where the jaw members 110 and 120 are aligned the left portion of the peripheral edge), the peripheral edge 121 includes an inner edge 121a that is configured to decrease current densities thereabout for either fusing, sealing, coagulating or fulgurating tissue during the first mode and an outer edge 121b that is configured to increase current densities thereabout for either dissecting, cutting, severing or transecting tissue during the second mode. With these purposes in mind, inner edge 121a includes a radius that is larger than a radius of the outer edge 121b, as best seen in FIG. 3B

As can be appreciated, in any of the foregoing alignment configurations of the jaw members 110 and 120, the peripheral edge 121 (and/or edges 121a and 121b) may include radii dimensioned to accommodate a specific surgical procedure, specific manufacturer preference, etc.

Operation of forceps 10 is described in terms of use of a method for electrosurgically treating tissue, such as, for example, during a hysterectomy, a colectomy and/or a Nissen fundoplication, commonly referred to in the art as a lap Nissen. Initially, the forceps 10 is inserted through an incision in a patient. Tissue is positioned between the jaw members 110 and 120. In the instance where a user wants to seal tissue, the user activates switch “S.” Activation of switch “S” indicates to the generator “G” and/or control system “CS” that the jaw members 110 and 120 are ready to operate in the bipolar mode of operation. Thereafter, generator “G” delivers electrosurgical energy to the respective seal plates 118 and 128 of the jaw members 110 and 120 to seal tissue positioned between the jaw members 110 and 120.

To dissect tissue, a user activates switch “D.” Activation of switch “D” indicates to the generator “G” and/or control system “CS” that the jaw members 110 and 120 are ready to operate in the monopolar mode of operation. A return pad or electrode may be positioned (at some time prior to operation of the forceps 10 in the monopolar mode) on the patient and functions as described above. Alternatively, an in some embodiments, the seal plate 118 may function as the return pad. In the monopolar mode of operation, generator “G” delivers electrosurgical energy to the seal plate 128 including the peripheral edge 121 and the distal tip 123 to dissect the electrosurgically treated tissue. During dissection, the jaw members 110 and 120 may be in either the open or closed position. Moreover, any portion of the seal plate 128 and/or the peripheral edge 121 including the distal tip 123 may be utilized to dissect the electrosurgically treated tissue.

For example, and in one particular surgical scenario, the jaw members 110 and 120 may be in the open position and the distal tip 123 may utilized to dissect the electrosurgically treated tissue. In this instance, the distal tip 123 is positioned adjacent tissue and moved in a direction indicated by directional arrow “M” into the tissue with a force of suitable proportion while simultaneously energizing the seal plate 128 (FIG. 2).

In another surgical scenario, the jaw members 110 and 120 may be in the open position and seal plate 128 may be utilized to dissect the electrosurgically treated tissue. In this instance, the seal plate 128 is positioned adjacent tissue and moved in a direction indicated by directional arrow “N” across the tissue with a force of suitable proportion while simultaneously energizing the seal plate 128 (FIG. 4).

In yet another surgical scenario, the jaw members 110 and 120 may be, initially, in the open position and seal plate 128 may utilized to dissect the electrosurgically treated tissue. In this instance, the seal plate 128 is positioned adjacent tissue and moved in a direction indicated by directional arrow “O” across the tissue with a force of suitable proportion while simultaneously energizing the seal plate 128 and closing the jaw members 110 and 120 (FIG. 5).

The forceps 10 including the jaw members 110 and 120 overcome some of aforementioned shortcomings of the above-referenced electrosurgical and/or ultrasonic instruments. More particularly, providing the forceps 10 with the finer seal plate 128 having the peripheral edge 121 eliminates the need for a knife blade and components associated therewith to dissect tissue. As can be appreciated, this lowers manufacturing costs of the forceps 10 and/or decreases or eliminates the manufacturing tolerances that are typically associated with conventional forceps. Moreover, while not discussed in great detail, the shaft 12 may be configured to bend or articulate; this provides a surgeon with greater flexibility with respect to treating and/or dissecting tissue when compared to ultrasonic instruments.

From the foregoing and with reference to the various figure drawings, those skilled in the art will appreciate that certain modifications can also be made to the present disclosure without departing from the scope of the same. For example, in certain embodiment it may prove useful to have one or both of the seal plates 118 and 128 with a textured or otherwise treated seal surface.

It is contemplated that rather than configuring one of the seal plates 118 and 128 to dissect tissue, a separate or additional device may be utilized to dissect tissue. For example, one or both of the jaw members 110 and 120 may include a second or auxiliary conductive surface. More particularly, a conductive surface (not shown) of suitable proportion may be operably disposed on one or both of an exterior surface of the jaw housing 117 and 127. For example, a conductive surface may extend along a length of a bottom exterior surface of the jaw housing 127 or a conductive surface may extend along a length of a top exterior surface of the jaw housing 117. As can be appreciated, in either of these instances, the conductive surface is configured to function substantially similar to that of the seal plate 128 described above.

It is contemplated that the generator “G” may be configured to automatically detect when to place the forceps 10 in either the first or second modes of operation. In this instance, switches 60 may be utilized in a limited capacity or eliminated altogether.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. An end effector assembly for an electrosurgical instrument, comprising:
a pair of first and second jaw members including respective seal plates having a width, the seal plates adapted to connect to an energy source, at least one of the first and second jaw members movable relative to the other jaw member from an open position, wherein the first and the second jaw members are disposed in spaced relation relative to one another, to a clamping position, wherein the first and second jaw members cooperate to grasp tissue therebetween, the first and second jaw members operable in two modes of operation, a first mode of operation for treating tissue and a second mode of operation for separating tissue,
wherein the width of the seal plate of the first jaw member is smaller than the width of the seal plate of the second jaw member such that in the second mode of operation the seal plate of the first jaw member is independently activatable from the seal plate of the second jaw member to facilitate the separation of tissue when the first and second jaw members are in one of the open and clamping positions.

2. An end effector assembly according to claim 1, wherein the first mode of operation includes one of fusing, sealing, coagulating and fulgurating tissue and the second mode of operation includes one of dissecting, cutting, severing and transecting tissue.

3. An end effector assembly according to claim 1, wherein the seal plate of the first jaw member includes a width that ranges from about 1 mm to about 3.4 mm and the seal plate of the second jaw member includes a width that ranges from about 3.5 mm to about 5 mm.

4. An end effector assembly according to claim 1, wherein the seal plate of the first jaw member includes a peripheral edge extending along a side surface of the first jaw member to a distal tip thereof, the peripheral edge including the distal tip configured to separate tissue upon actuation when the first and second jaw members are in one of the open and closed position.

5. An end effector assembly according to claim 1, wherein the first and second jaw members are centrally aligned along a common axis.

6. An end effector assembly according to claim 4, wherein the first and second jaw members are aligned along the peripheral edge of the seal plate of the first jaw member.

7. An end effector assembly according to claim 6, wherein the peripheral edge includes an inner edge configured to decrease current densities thereabout for one of fusing, sealing, coagulating and fulgurating tissue during the first mode of operation and an outer edge configured to increase current densities thereabout for one of dissecting, cutting, severing and transecting tissue during the second mode of operation.

8. An end effector assembly according to claim 7, wherein the inner edge includes a radius that is larger than a radius of the outer edge.

9. An end effector assembly according to claim 1, wherein the second jaw member includes at least one insulative standoff configured to maintain a specific gap distance between the first and second jaw members when the first and second jaw members are in the clamping position.

10. An end effector assembly according to claim 9, wherein the at least one insulative standoff is operably disposed at a distal end of the second jaw member and is configured to contact a distal tip of the first jaw member when the first and second jaw members are in the clamping position.

11. A system for performing an electrosurgical procedure, comprising:
an energy source configured to function in at least two modes of operation, a first mode of operation for treating tissue and a second mode of operation for dissecting tissue;
an electrosurgical instrument comprising:
a handle having at least one shaft that extends therefrom that defines a longitudinal axis therethrough; and
an end effector assembly operatively connected to a distal end of the at least one shaft and having a pair of first and second jaw members including respective seal plates having a width, the seal plates adapted to connect to an energy source, at least one of the first and second jaw members movable relative to the other jaw member from an open position, wherein the first and the second jaw members are disposed in spaced relation relative to one another, to a clamping position, wherein the first and second jaw members cooperate to grasp tissue therebetween, the first and second jaw members operable in two modes of operation, a first mode of operation for treating tissue and a second mode of operation for separating tissue,
wherein the width of the seal plate of the first jaw member is smaller than the width of the seal plate of the second jaw member such that in the second mode of operation the seal plate of the first jaw member is independently activatable from the seal plate of the second jaw member to facilitate the separation of tissue when the first and second jaw members are in one of the open and clamping positions.

12. A system for performing an electrosurgical procedure according to claim 11, wherein the energy source is a single electrosurgical generator that is configured to operate in bipolar and monopolar modes of operation.

13. A system for performing an electrosurgical procedure according to claim 12, wherein an actuation device is operably associated with one of the electrosurgical generator and the electrosurgical instrument and is configured to switch the electrosurgical instrument between the first and second modes of operation.

14. A system for performing an electrosurgical procedure according to claim 13, wherein the actuation device is operably disposed on the handle of the electrosurgical instrument and includes a first switch for manually placing the electrosurgical instrument into the first mode of operation and a second switch for manually placing the electrosurgical instrument into the second mode of operation.

15. A method for performing an electrosurgical procedure, comprising:
positioning tissue between first and second jaw members of an electrosurgical instrument, each of the first and second jaw members including respective seal plates having a width, the respective seal plates adapted to connect to an energy source, the first and second jaw members operable in first and second modes of operation, wherein the width of the seal plate of the first jaw member is smaller than the width of the seal plate of the second jaw member;
closing the first and second jaw members such that the tissue is clamped therebetween;
transmitting electrosurgical energy in the first mode of operation to the first and second jaw members for electrosurgically treating tissue; and
transmitting electrosurgical energy in a second mode of operation to the jaw member with the seal plate with the smaller width for dissecting the electrosurgically treated tissue.

16. A method according to claim 15, wherein the step of positioning includes the step of providing in the second mode of operation that the seal plate of the first jaw member is independently activatable from the seal plate of the second jaw member to facilitate the separation of tissue when the first and second jaw members are in one of the open and clamping positions.

17. A method according to claim 15, wherein the first mode of operation includes one of fusing, sealing, coagulating and fulgurating tissue and the second mode of operation further includes one of cutting, severing and transecting tissue.

18. A method according to claim 15, wherein the step of positioning includes the step of providing the seal plate of the first the jaw member with a width that ranges from about 1 mm to about 3.4 mm and the seal plate of the second jaw member with a width that ranges from about 3.5 mm to about 5 mm.

19. A method according to claim 15, wherein the seal plate of the first jaw member includes a peripheral edge extending along a side surface of the first jaw member to a distal tip thereof, the peripheral edge including the distal tip configured to separate tissue upon actuation when the first and second jaw members are in one of the open and closed position.

20. A method according to claim 15, wherein the step of positioning includes the step of providing first and second jaw members that are aligned along the peripheral edge of the seal plate of the first jaw, wherein the peripheral edge includes an inner edge configured to decrease current densities thereabout for one of fusing, sealing, coagulating and fulgurating tissue during the first mode of operation and an outer edge configured to increase current densities thereabout for one of dissecting, cutting, severing and transecting tissue during the second mode of operation.
